(19) United States
(12) Patent Application Publication
(10) Pub. No.: US 2019/0105484 A1
(43) Pub. Date: Apr. 11, 2019

(54) MALE TO FEMALE COUPLING

(71) Applicant: NEOMED, INC., Woodstock, GA (US)

(72) Inventors: David A. DOORNBOS, Woodstock, GA (US); Benjamin M. DAVIS, Woodstock, GA (US)

(73) Assignee: NEOMED, INC., Woodstock, GA (US)

(21) Appl. No.: 16/154,479

(22) Filed: Oct. 8, 2018

Related U.S. Application Data
(60) Provisional application No. 62/568,832, filed on Oct. 6, 2017.

(51) Int. Cl. A61M 39/10; A61J 15/00

(52) U.S. Cl. CPC A61M 39/1011; A61M 2202/0482; A61M 2039/1077; A61J 15/0026

(57) ABSTRACT

A male to female coupling for attachment between a connector of an enteral fluid delivery assembly and a connector of a syringe. In example embodiments, the male to female coupling comprises an annular space for collection of unused feeding fluids.

### CROSS-REFERENCE TO RELATED APPLICATION

This application claims the benefit of U.S. Provisional Patent Application Ser. No. 62/568,832 filed Oct. 6, 2017, the entirety of which is hereby incorporated herein by reference for all purposes.

### TECHNICAL FIELD

The present invention relates generally to the field of neonatal and enteral feeding systems and components, and more particularly to a coupling for use in enteral fluid delivery applications.

### BACKGROUND

Neonates and other healthcare patients are often administered fluids such as medications, nutritional fluids and supplements via enteral fluid delivery, commonly utilizing delivery systems including fluid containers, syringes, feeding tubes and other components. These components are often interconnected by connectors or couplings such as Luer connectors, or the more recently developed ENFit connector (ISO Standard 80369).

In some embodiments, these enteral connectors or couplings may include outer housing geometries with recesses or areas that could retain small quantities of unused feeding fluids that might allow for bacteria colonization or contain other potential contaminants. For example, as shown in FIGS. 1-2, a connector C of a feeding tube, feeding tube extension set or the like is connected with a connector SC of a syringe for the enteral delivery of nutritional or medicinal fluids. The connector C has a threaded outer collar W with a closed endwall that allows for the containment of unused feeding fluids F within an annular well or recess R thereof. Among most common to occur during the disconnection of the connector SC of the syringe S, fluid F within a conduit L of a male hub H of the connector C tends to overflow from the male hub H and within the annular recess R (generally contained between the male hub H and the threads of the threaded outer collar W). Typically, the conduit extending between the connector C and connector SC can comprise a vacuum or generally become pressurized, for example, such that the initial disconnection therebetween generally relieves the vacuum or pressure present, and wherein relieving the vacuum or pressure generally causes at least some unused or residual feeding fluids to overflow or generally become exposed to the atmosphere, some of which can enter into one or more recesses or areas of the components (e.g., the annular recess R).

Exposure of the fluid retained within the annular recess R to the surrounding environment presents possible contamination issues, and subsequent reconnection of the connector SC of the syringe S may cause the potentially contaminated fluid to communicate within the fluid passage or lumen L, thereby potentially exposing a patient to contaminated fluids, bacteria, or other unwanted contaminants. As shown in FIG. 2, it is also possible for fluids to leak from the syringe connector and within the recessed area as shown.

As depicted in FIGS. 3-4, a vented enteral connector VC can be used instead of the connector C, so as to reduce the likelihood of fluids F collecting in the annular recess R. For example, a pair of vents V extend through the endwall and assist in facilitating the drainage of any fluid that enters the recessed area. U.S. patent application Ser. No. 14/844,956, which is incorporated herein by reference, discloses a vented male ENFit enteral coupling or connector having a housing structure with drainage passages or vents to eliminate or reduce the likelihood of retaining feeding liquids or other contaminants in the outer housing. As shown in FIG. 4, fluids F similarly overflow from the male hub H and within the annular recess (e.g., the area between the male hub H and the threaded outer collar W) during disconnection of the connector SC from the vented connector VC. However, due to the endwall being vented, fluids F do not collect in the annular recess but instead drain from the vents V of the vented connector VC. While most all of the contaminated fluids are prevented from being retained in the recessed area, fluid residue and other smaller amounts of fluid may remain within the recessed area, and thus, bacteria colonization or the containment of other potential contaminants may be present. In some cases, a cleaning tool or device can be used to clean the areas of the vented connector VC that are exposed to the fluids. U.S. patent application Ser. No. 15/009,073 is incorporated herein by reference and discloses a cleaning device for cleaning a fluid transfer connector, for example, which can be a vented male ENFit enteral connector as described above.

Further improvements in the field are desirable, and it is to the provision of a male to female coupling for enteral couplings or connectors that the present invention is primarily directed.

### SUMMARY

In example embodiments, the present invention provides a male to female coupling for attachment between a connector and a syringe. In example embodiments, the coupling is configured for coupling engagement between a male ISO 80369-3 formatted coupling of a feeding tube connector or enteral fluid delivery connector and a female ISO 80369-3 formatted coupling of a syringe.

In one aspect, the present invention relates to a male to female enteral coupling including a body extending from a first end to a second end. The first end includes a male hub surrounded by an outer cylindrical collar, an annular space defined between the male hub and the outer cylindrical collar, and a lumen defined within the male hub. The second end includes a female connector having a generally cylindrical body, and a receiver defined within the body and in communication with the lumen, wherein the receiver is configured for receiving a male connector. In example embodiments, the annular space is configured for collecting fluids discharged or overflowing during a post-delivery disconnection or separation of a fluid delivery syringe and an enteral fluid delivery device connector.

In example embodiments, the male to female coupling is configured for interconnection between the fluid delivery syringe and an enteral fluid delivery device connector. In example embodiments, the first end is configured for engagement with a syringe connector of the fluid delivery syringe and the second end is configured for engagement with a male hub of the enteral fluid delivery device connector. In example embodiments, the first end comprises a male ISO 80369-3 compatible coupling. In example embodiments, the second end comprises a female ISO 80369-3 compatible coupling. In example embodiments, the coupling further includes a flange extending from at least a portion of the body. In example embodiments, the flange includes an outwardly-protruding member, and wherein a width defined between the body and an outermost portion of the outwardly-protruding member is a minimum of about 1.25 inches. In example embodiments, the hub further includes a tether for maintaining engagement of the body with the enteral fluid delivery device connector. In example embodiments, the coupling further includes a lumen extension tip centrally positioned within the receiver of the cylindrical body, the lumen extension tip having a conduit defined therein and in communication with the lumen of the male hub of the first end. In example embodiments, the coupling further includes a skirt projecting from the body and towards the second end, at least a portion of the skirt surrounding the cylindrical body of the second end. In example embodiments, the coupling further includes one or more resilient clips formed with at least a portion of the skirt.

In another aspect, the present invention relates to a disposable enteral fluid delivery connector for interconnection between a fluid delivery syringe and a connector of an enteral feeding tube. The disposable enteral fluid delivery connector a body extending along a longitudinal axis from a first end to a second end, a hub and an outer cylindrical collar defined at the first end, and a receiver having a cylindrical body at the second end. An annular space is defined between the hub and the outer cylindrical collar, and the annular space terminates at an endwall, and the hub is configured for compatible engagement with the fluid delivery syringe. A lumen extends through the cylindrical body and hub so as to provide fluid communication through the body, and the receiver is configured for compatible engagement with the connector of the enteral feeding tube. In example embodiments, with the body interconnected between the fluid delivery syringe and the connector of the enteral feeding tube, the fluid delivery syringe is disconnected from the hub of the first end such that any fluids discharged from the lumen of the hub or from the fluid delivery syringe is collected in the annular space defined at the first end of the body so as to prevent exposure of fluids to the connector of the enteral feeding tube.

In example embodiments, the first end includes a male ISO 80369 compatible coupling. In example embodiments, the second end includes a female ISO 80369 compatible coupling. In example embodiments, the coupling further includes a flange extending from at least a portion of the body. In example embodiments, the flange includes an outwardly-protruding member, and wherein a width defined between the body and an outermost portion of the outwardly-protruding member is a minimum of about 1.25 inches. In example embodiments, the coupling further includes a tether for maintaining engagement of the body with the enteral fluid delivery device connector. In example embodiments, the coupling further includes a lumen extension tip centrally positioned within the receiver of the cylindrical body, the lumen extension tip comprising a conduit defined therein and in communication with the lumen of the male hub of the first end. In example embodiments, the coupling further includes a skirt projecting from the body and towards the second end, at least a portion of the skirt surrounding the cylindrical body of the second end. In example embodiments, the coupling further includes one or more resilient clips formed with at least a portion of the skirt, the one or more resilient clips being configured for removable or permanent engagement with the connector of the enteral feeding tube.

In yet another aspect, the present invention relates to a method of connecting and disconnecting enteral feeding connectors to prevent contamination of components. In example embodiments, the method includes providing a fluid delivery device, the fluid delivery device having a connector; providing an enteral feeding tube assembly, the assembly including an elongate tube having at least one connector; providing a male to female coupling, the male to female coupling having a body extending along a longitudinal axis from a first end to a second end, a hub and an outer cylindrical collar defined at the first end and a receiver having a cylindrical body at the second end, wherein an annular space is defined between the hub and the outer cylindrical collar, the annular space terminating at an endwall and the hub being configured for compatible engagement with the fluid delivery syringe, and wherein a lumen extends through the cylindrical body and hub so as to provide fluid communication through the body, the receiver of the second end configured for compatible engagement with the at least one connector of the enteral feeding tube assembly; connecting the receiver of the male to female coupling with the at least one connector of the enteral feeding tube assembly; connecting the hub of the male to female coupling with the connector of the fluid delivery device; discharging fluid from the fluid delivery device, through the lumen of the male to female coupling, and through the conduit of the enteral feeding tube assembly; disconnecting or separating the connector of the fluid delivery device from the hub of the male to female connector, wherein any fluids overflowing from the lumen of the hub of the male to female coupling are retained within the annular space; and disconnecting the receiver of the second end of the male to female coupling from the at least one connector of the enteral feeding tube assembly.

In another aspect, a method of enteral feeding including providing a fluid delivery device; providing an enteral feeding tube assembly, the assembly including an elongate tube having at least one connector; providing a male to female coupling, the male to female coupling having a body extending along a longitudinal axis from a first end to a second end; connecting the second end of the male to female coupling with the at least one connector of the enteral feeding tube assembly; and connecting the first end of the male to female coupling with the fluid delivery device.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of example embodiments are explanatory of example embodiments of the invention, and are not restrictive of the invention, as claimed.

### BRIEF DESCRIPTION OF THE DRAWINGS

FIG. 1 shows a cross-section of a prior art connector connected to a connector of a syringe.

FIG. 2 shows the cross-section of the prior art connector of FIG. 1, with the prior art connector disconnected from the connector of the syringe and showing the collection of unused feeding fluids within a portion of the connector.

FIG. 3 shows a cross-section of another prior art connector connected to a connector of the syringe, and wherein the prior art connector has a pair of vents formed in an endwall of a cylindrical collar so as to facilitate drainage of unused feeding fluids that may be retained within a portion of the connector.

FIG. 4 shows the cross-section of the prior art connector of FIG. 3, with the prior art connector disconnected from the connector of the syringe and showing the drainage of the unused feeding fluids from the vents of the endwall of the cylindrical collar.

FIG. 5 shows a cross-section of a male to female coupling according to an example embodiment, wherein the male to female coupling is interconnected between the connector and syringe connector of FIG. 3.

FIG. 6 shows the cross-section of FIG. 5, with the connector of the syringe disconnected from the male to female coupling and showing the collection of unused fluids within an annular recess thereof.

FIG. 7 shows the cross-section of FIG. 6, with the male to female coupling further disconnected from the connector.

FIG. 8 shows an cross-sectional assembly view of a male to female hub positioned for connection between a connector of a syringe and another connector according to another example embodiment of the present invention, wherein the connector of the syringe an a connector of the male to female coupling include a lumen extension tip.

FIG. 9 shows a cross-sectional assembly view of a male to female hub positioned for connection between a connector of a syringe and another connector according to another example embodiment of the present invention, showing the male to female coupling and a closure or cap being tethered to the connector.

FIG. 10 shows a perspective view of a male to female coupling according to another example embodiment of the present invention.

FIG. 11 is a cross-sectional view of the male to female coupling of FIG. 10 taken along line 11-11.

FIG. 12 shows a cross-sectional view of a male to female coupling according to another example embodiment of the present invention.

FIG. 13 shows a cross-sectional view of a male to female coupling according to another example embodiment of the present invention.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

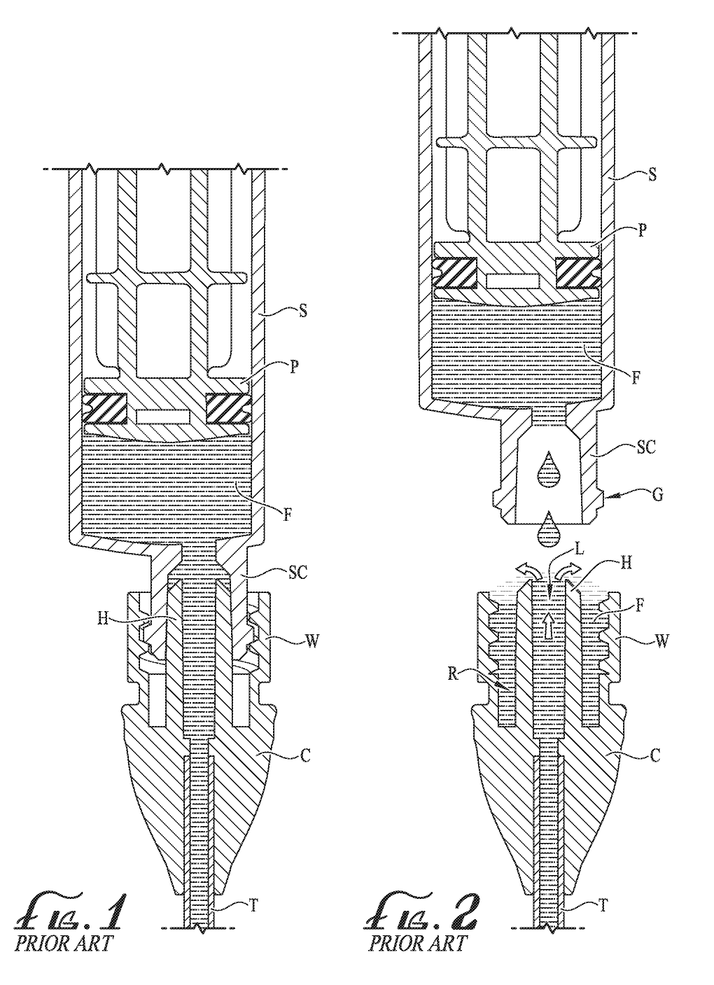

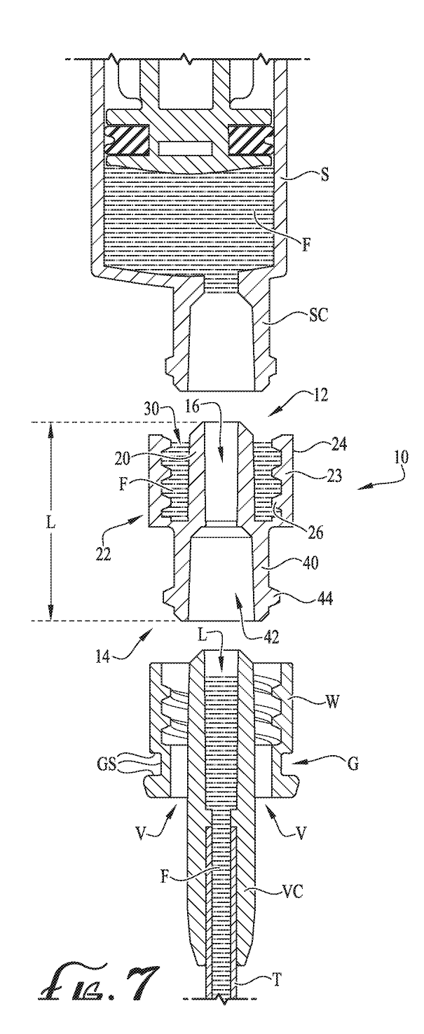

With reference now to the drawing figures, wherein like reference numbers represent corresponding parts throughout the several views, FIGS. 5-7 show a male to female hub connector or coupling 10 according to an example embodiment of the present invention. In example embodiments, the male to female coupling 10 is intended to be interconnected between enteral connectors or couplings, for example, such that unused feeding fluids or other bacterial colonization and contamination is entirely eliminated. For example, in comparison to the embodiments of FIGS. 1-4, rather than connecting the syringe coupling SC of the syringe S directly with the connector C and vented connector VC, the male to female coupling 10 is interconnected therebetween so as to prevent any fluids F within the syringe S (or conduit(s) of the connectors) from becoming exposed to any portion of the connector or vented connector C, VC.

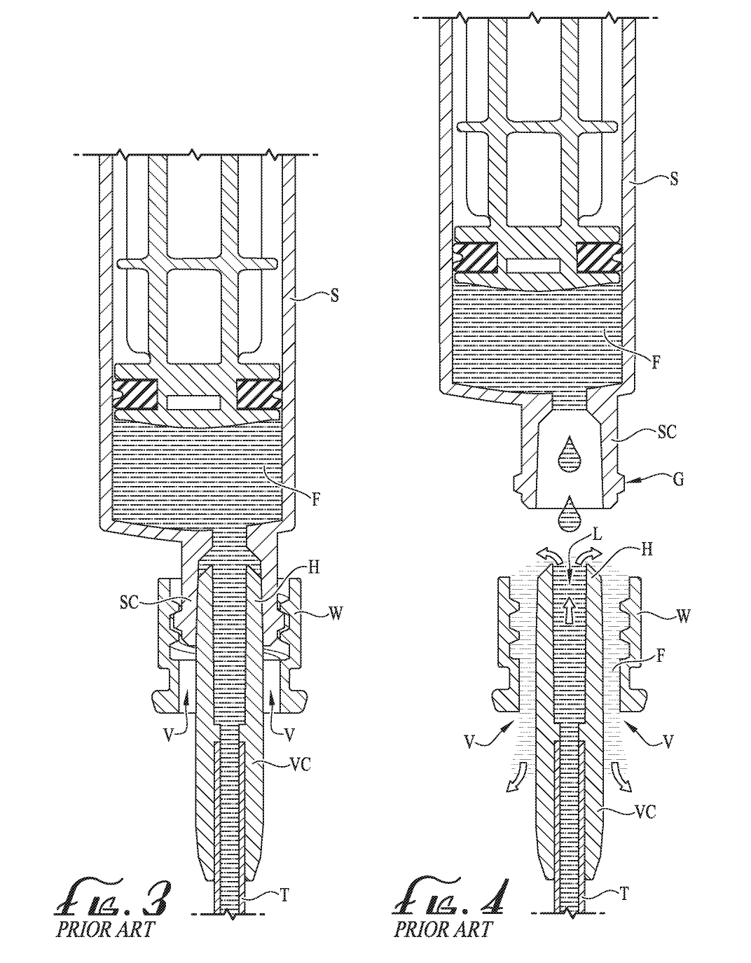

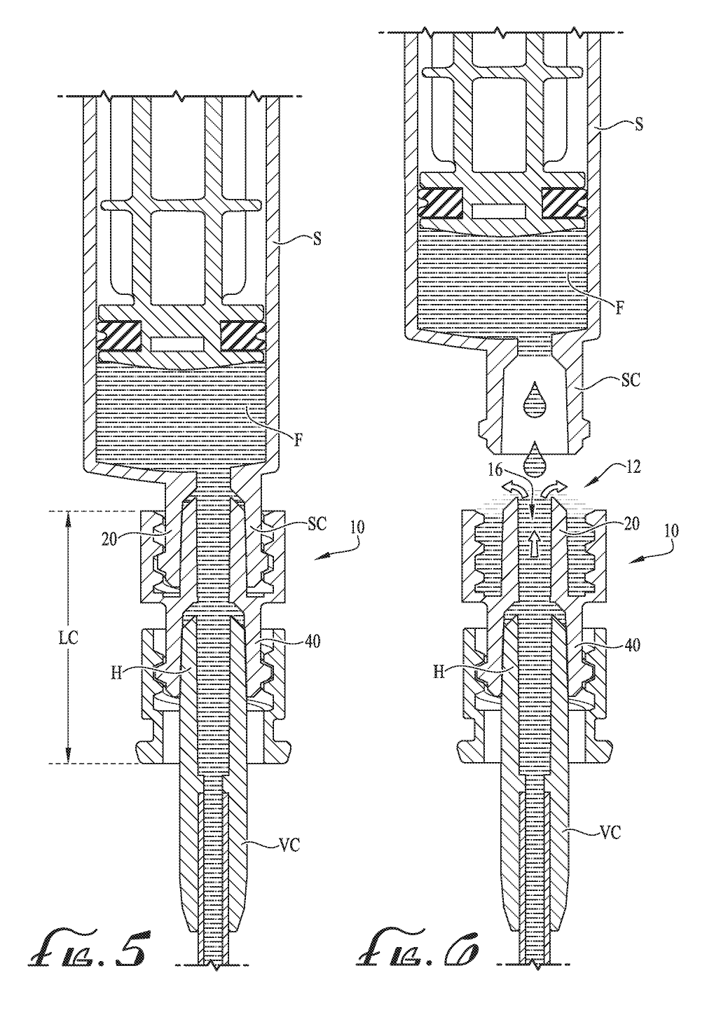

As will be described herein and shown in FIGS. 5-9, the vented connector VC is provided whereby a tube, conduit or other member comprising a fluid passageway defined therein is connected therewith for the furtherance of the fluid F to a patient for enteral delivery. According to example embodiments, the vented connector VC and tube T connected therewith is an extension set for connection and fluid communication with a feeding tube. According to other example embodiments of the present invention, the vented connector VC and tube T can be a feeding tube, or can otherwise be an enteral component for facilitating the delivery of nutritional or medicinal fluids. In other example embodiments, the vented connector VC need not be vented, for example, as is shown in FIGS. 1-2. Thus, according to example embodiments of the present invention, while FIGS. 5-9 show a vented connector VC and connected tube T of an extension set for enteral delivery, the connector C or other enteral connectors and tubes or conduits can be used as desired. Furthermore, as will be referenced throughout the figures, the vented connector VC is one of many components or connectors that can be used with the present invention. Accordingly, the vented connector VC is termed as an enteral fluid delivery connector VC, which can comprise the connector C (see FIGS. 1-2), the vented connector VC (see FIGS. 3-4), an enteral feeding tube assembly, a feeding tube extension set, an enteral feeding tube, or can comprise other enteral couplings or connectors as desired.

As depicted in FIGS. 5-6, the coupling 10 is interconnected between the syringe connector SC and the enteral fluid delivery connector VC so that fluids F discharged from the syringe coupling SC flow through the conduits of the coupling 10, the enteral fluid delivery connector VC and through the tube T for delivery to the patient. After fluid delivery has completed, or when it is desired to cease fluid delivery, the syringe S is disconnected by disengagement of the syringe connector SC with a hub 16 of the coupling 10. Similarly to the embodiment of FIGS. 1-2, fluid F within a conduit 16 of the male hub 20 of the coupling 10 may overflow from the male hub 20 and within an annular recess 30 (generally contained between the male hub 20 and threads 26 of the threaded outer collar 23). The coupling 10 can then be disconnected from the enteral fluid delivery connector VC without causing/allowing any of the unused fluids F from being exposed to the enteral fluid delivery connector VC (and more specifically exposed in the annular recess between the hub H and the internally threaded portion of the outer collar W).

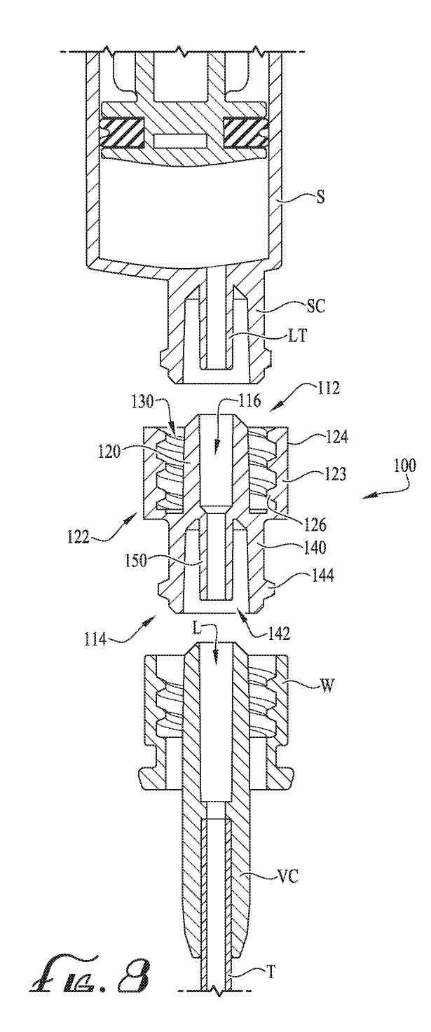

According to some example embodiments, the enteral fluid delivery connector VC and/or the coupling 10 (and during the separation thereof) can be reoriented, for example, such that they are generally not oriented in a vertical manner as depicted in FIGS. 6-7. For example, according to example embodiments, any fluids remaining within the lumen 16 after disconnection of the syringe connector SC and prior to separation of the coupling 10 and enteral fluid delivery connector VC may be drained or otherwise removed therefrom (e.g., by reorientation of the coupling 10 and enteral fluid delivery connector VC) prior to complete separation of the coupling 10 and the enteral fluid delivery connector VC. In other example embodiments, disconnection of the syringe connector SC from the hub 20 alleviates a vacuum within the fluid path/conduit such that about the entirety of the fluid within the conduit 16 is generally removed therefrom or escapes prior to the separation of the coupling 10 and enteral fluid delivery connector VC, and thus, the coupling 10 and enteral fluid delivery connector VC can be disconnected without any concern of fluid F overflowing from the hub H of the enteral fluid delivery connector VC and within the annular recess between the hub H and the outer cylindrical collar W.

For example, as depicted in FIG. 7, the level of fluid F within the lumen L of the enteral fluid delivery connector VC after the coupling 10 is disconnected therefrom is generally below an end of the hub H, and due to any vacuum or pressurization of the fluid path being alleviated by disconnection of the syringe coupling SC and hub 20, fluid F within the lumen L does not behave as described above with respect to FIGS. 1-4, and thus, fluid F does not overflow from the hub H and within the annular recess, thereby entirely eliminating the possibility of unused fluids being exposed and becoming contained within portions of the enteral fluid delivery connector VC. Accordingly, the coupling 10 preferably provides a contamination-free enteral fluid delivery solution so as to entirely eliminate the risk of contamination of the enteral fluid delivery connector VC, especially during the connection and disconnection of a fluid delivery device (e.g., syringe) so as to prevent any fluids F from being contained within one or more portions of the enteral fluid delivery connector VC. For example, according to example embodiments of the invention, the enteral fluid delivery connector VC and connected tube T is an enteral fluid delivery extension set, which can be reusable for up to a period of time before being disposed. As such, by using the coupling 10 as an intermediate connector between the syringe connector SC and the enteral fluid delivery connector VC, the risk of the reusable enteral fluid delivery component becoming contaminated is substantially (if not entirely) eliminated. In example embodiments, the coupling 10 is intended for a single use, and then discarded thereafter. Thus, according to example embodiments, the coupling 10 is a disposable enteral fluid delivery coupling, for example, wherein hub and receiver thereof fluidly couple with the receiver of the syringe and a hub of the connector of the enteral fluid delivery assembly (e.g., comprising the enteral fluid delivery connector VC). In alternate example embodiments, the coupling can be reusable and/or cleanable. In some example embodiments, one or more couplings 10 can be placed in a cleaning solution or otherwise exposed to one or more forms of energy such that the one or more couplings 10 are sterilized for reuse.

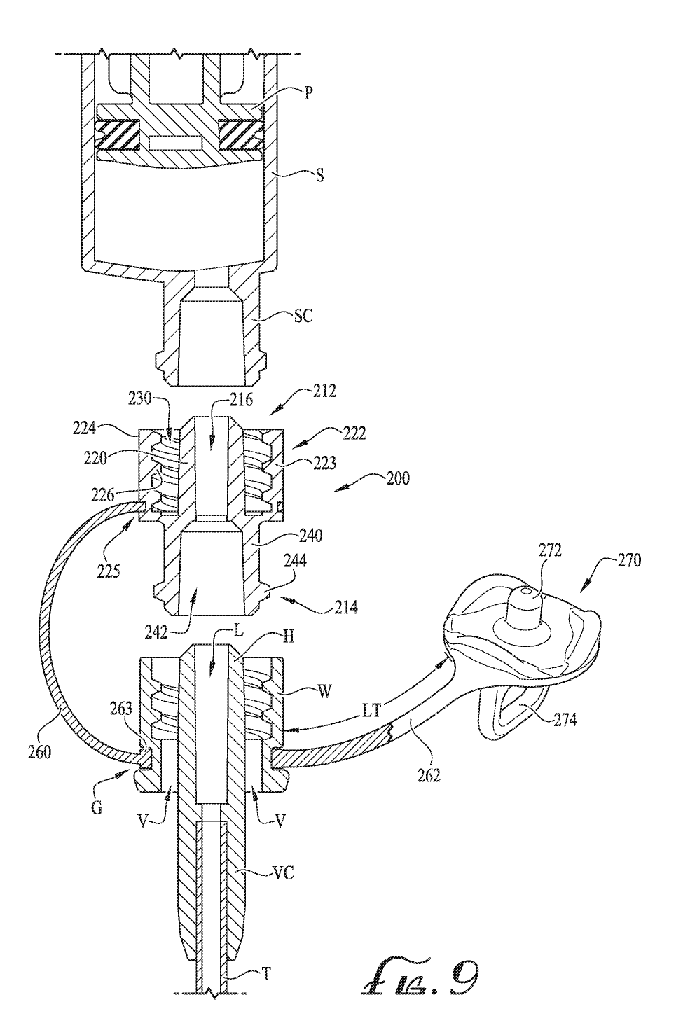

Thereafter, with the coupling 10 disconnected from the hub H of the vented connector (and typically discarded), a cap, plug or cover (see FIG. 9) can be attached to the vented connector such that the lumen L can be sealed from potential contamination. According to one example embodiment, the cap seals the lumen and covers at least a portion of the annular space defined between the hub and the outer cylindrical collar. U.S. patent application Ser. No. 14/844,956, which is incorporated herein by reference in its entirety, discloses a vented male ISO 80369-3 compatible enteral coupling or connector having a housing structure with drainage passages or vents to eliminate or reduce the likelihood of retaining feeding liquids or other contaminants in the outer housing, and that a cap having at least one vent opening can be tethered to the vented enteral coupling to provide for fluid drainage and airflow ventilation of the annular space when the cap is in the plugged position. Accordingly, a cap, plug or cover member can be provided with the enteral fluid delivery connector VC.

As depicted in FIG. 7, the male to female coupling 10 comprises a body extending along a longitudinal axis from a first end 12 to a second end 14. In example embodiments, the first end 12 of the body comprises a male hub 20 defining a lumen 16 extending therethrough, and the second end 14 comprises a female connector comprising a generally cylindrical body 40, and a receiver 42 defined within the body 40 and with the receiver 42 being in communication with the lumen 16. According to some example embodiments of the present invention, the first end 12 comprises a male ISO 80369-3 compatible or formatted enteral coupling, and the second end 14 comprises a female ISO 80369-3 compatible or formatted enteral coupling.

In example embodiments, the coupling 10 comprises a length L that is defined between an end portion of the hub 20 at the first end 12 and an end portion of the cylindrical body 40 at the second end 14. In example embodiments, the length L is between about 0.5-3.5 inches, for example between about 0.5-1.5 inches according to some example embodiments, and for example, about 0.67 inches according to one example embodiment. According to one example embodiment, the length L is 0.67 inches. According to another embodiment, the length L is a maximum of 0.67 inches. According to another example embodiment, the length L is a minimum of 0.67 inches. According to yet another example embodiment, the length L is 0.6 inches. In other example embodiments, the length L can between about 0.4-5 inches.

In example embodiments, an outer cylindrical collar 23 surrounds the male hub 20 and an annular space 30 is defined between the male hub 20 and the outer cylindrical collar 23. Preferably, as described above, any unused fluids F collect within the annular space 30, and thus, the annular space between the outer cylindrical collar W and the hub B of the enteral fluid delivery connector VC remains free from unused fluids throughout enteral fluid delivery, and thus maintains a clean and aseptic connector VC. Thus, according to some example embodiments, by using the coupling 10 as disclosed herein, rather than the connection of FIGS. 1-4, a cleaning tool is not necessarily needed, for example, as the annular space between the outer cylindrical collar W and the hub H remain free from fluids or other substances, thereby eliminating common concerns such as bacteria growth and contamination.

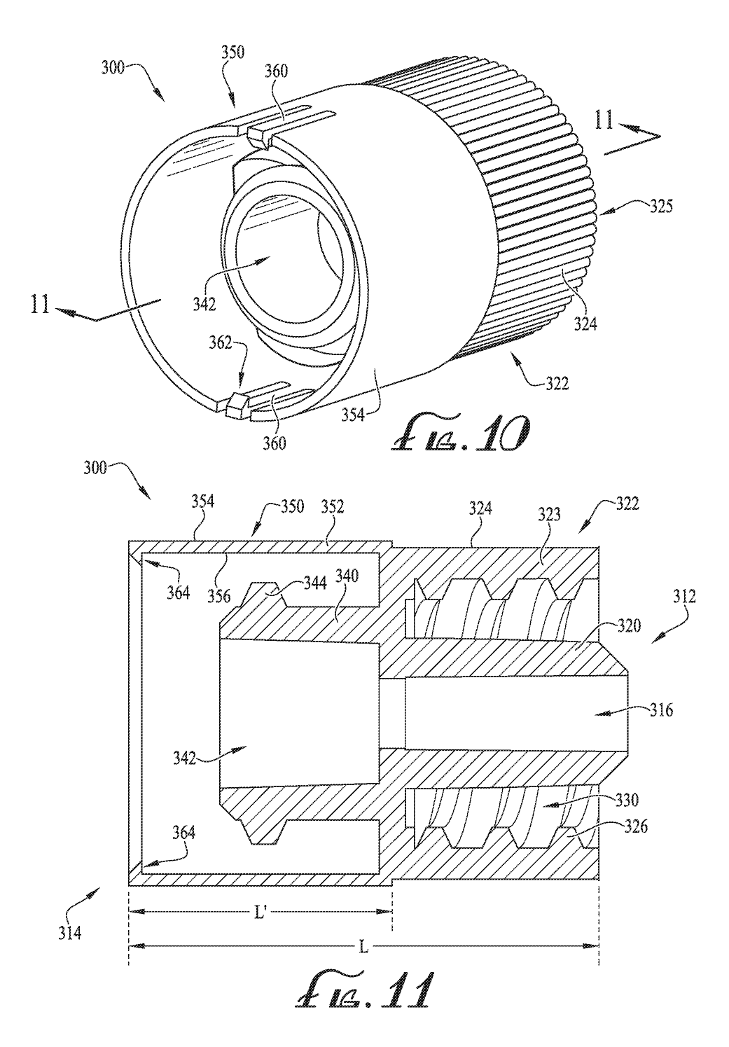

In example embodiments, the outer cylindrical collar 23 generally comprises threads 26 on an internal portion for providing engagement with one or more threads or lugs G of the syringe connector SC of the syringe S. In some example embodiments, an outer surface or periphery portions 24 of the collar 23 can comprise one or more surface features or other recesses, indentions, projections or other features such that an enhanced gripping surface can be provided to assist a user in attachment/detachment of the coupling 10 from the connector C or syringe S (for example, see 324 of FIG. 10).

According to another example embodiment, as depicted in FIG. 8, the syringe connector SC and/or a male to female coupling 100 can comprise a lumen extension tip LT, 150, or for example, a low-dose tip comprising a small diameter lumen, for example, such that the overflow of unused feeding fluids is further reduced (if not entirely eliminated) and wherein the volume of the conduit is reduced to a volume that is substantially similar to the volume of the conduit when the syringe is connected directly to the feeding tube connector (see FIGS. 1-4). For example, the syringe S comprises a lumen extension tip LT and the coupling 100 comprises a lumen extension tip 150, both of which are centered within the receiver of the cylindrical collars and configured to occupy at least a portion of the lumen 116, L of the male hubs 120, H, thereby substantially reducing the volume of the conduit. U.S. patent application Ser. No. 15/210,282 filed Jul. 14, 2016 and U.S. patent application Ser. No. 15/659,323 filed Jul. 25, 2017 (2N11.1-373 & 2N11.1-432) disclose syringes and couplings/connectors comprising lumen extension tips, the entirety of which is incorporated by reference herein in its entirety.

According to another example embodiment as depicted in FIG. 9, a coupling 200 is provided for interconnection between the syringe S and the enteral fluid delivery connector VC. According to example embodiments, a first tether 260 is provided for attachment of the coupling 200 to the enteral fluid delivery connector VC, and a second tether 262 is provided for attachment of the cap 270 to the enteral fluid delivery connector VC. Accordingly, in example embodiments, the coupling 200 is tethered to the enteral fluid delivery connector VC, which is indirectly tethered to the cap 270. In example embodiments, a groove or generally cylindrical recess 225 is provided for receiving an end of the tether 260 (e.g., a retaining ring or the like), and a generally opposite end of the tether 260 comprises a retaining ring for attachment to a groove or annular channel G of the enteral fluid delivery connector VC. Preferably, the coupling 200 can be connected/disconnected from the enteral fluid delivery connector VC while remaining engaged with the tether. According to some example embodiments, when desired, the coupling 200 can be removed from attachment with the tether 260, for example, when it is desired to dispose of the coupling 200 after use. In another example embodiment, an unused coupling can be reconnected to the tether 260 to be used during the next enteral fluids delivery. Thus, according to example embodiments, the tether 260 is preferably provided for removable/permanent attachment of the coupling 200 to the enteral fluid delivery connector VC so as to not pose a choking hazard, for example, so that when the coupling is disconnected from the enteral fluid delivery connector VC, the coupling 200 remains attached to at least a portion of the enteral fluid delivery connector VC until it is desired to be discarded. According to some example embodiments, the tether 260 is configured to be breakable, or for example, can easily be cut or torn so that the coupling 200 can be discarded. According to some example embodiments, the enteral fluid delivery connector VC comprises both a coupling 200 and cap 270 tethered thereto. And after the first fluid delivery, the coupling 200 can be removed from attachment (via tether) with the enteral fluid delivery connector VC, and a new unused coupling 200 (and tether) can be attached thereto for use. In example embodiments, the tethers 260, 262 need not have retaining rings, but can comprise other engagement members for permanent/removable attachment with coupling 200 and the enteral fluid delivery connector VC. According to other example embodiments, the tethers 260, 262 are generally integrally formed with the coupling 200 and the enteral fluid delivery connector VC.

The cap 270 comprises body defining one or more vent openings, a plug for sealing the lumen of the hub, and a handle 274 for gripping to move the cap between its unplugged and plugged positions. In example embodiments, the tether 262 extends from the body of the cap 270 to a retaining ring or other engagement member that is connected with the groove of the enteral fluid delivery connector VC. In example embodiments, the tether 262 is sized so as to prevent its use while the coupling 200 is connected with the enteral fluid delivery connector VC, but allow its use for sealing the lumen L of the enteral fluid delivery connector VC when the coupling 200 is disconnected from the hub of the enteral fluid delivery connector VC. For example, the tether 262 comprises a length LT that is generally shorter than a length LC defined between the outermost portions of the collars (see FIG. 5). In other example embodiments, the length LC is defined between an outermost portion of the collar 23 of the coupling 10 and a midpoint of the groove G that is defined within a portion of the enteral fluid delivery connector VC. Thus, according to example embodiments, rather than being able to keep the coupling 200 connected with the hub of the enteral fluid delivery connector VC and seal it with the plug 272 of the cap 270, the clinician is forced to remove the coupling 200 so that the lumen L of the enteral fluid delivery connector VC can be sealed (e.g., due to the length LT of the tether 262 being too short for plugging the lumen 216 of the hub 220). According to some example embodiments, the length LT of the tether 262 is generally between about 0.5-0.9 inches. According to another example embodiment, the length LT is between about 0.4-1.25 inches. Optionally, the length LT of the tether 262 can be sized as desired.

According to another example embodiment, the male to female coupling comprises a tethered plug coupled thereto, for example, to comply with 16 CFR § 1501.4 (e.g., not entirely fitting within the test cylinder specified by § 1501.4) and to provide for plugging the lumen or conduit of the hub after use. In some example embodiments, the coupling comprises two tethered plugs, for example, wherein one of the plugs is provided for sealing the lumen of the hub of the first end of the coupling and the other plug is provided for sealing the lumen L of the hub H of the enteral fluid delivery connector VC. According to some example embodiments, the plug is configured for permanent engagement with the first end of the male to female coupling, and thus, prevents a clinician or user from reusing the male to female coupling multiple times, for example, wherein after its first use, the permanent cap would be attached to the first end, and thus, the coupling would be replaced with a new, unused coupling before the second feeding or administration of medicine. According to another example embodiment of the present invention, a tethered plug can be provided for permanent attachment to the hub of the coupling. For example, according to some example embodiments, the plug is resilient and at least partially deformable, for example, such that at least a portion thereof can be inserted through the entirety of the lumen of the hub and become permanently caught within the receiver of the second end. Thus, after its use, the syringe connector and enteral fluid delivery connector VC are disconnected from the coupling, and a plug permanently engages with the coupling to prevent reuse, for example, wherein the plug is inserted through the entirety of the lumen until becoming permanently engaged therein, for example, such that extension of the plug beyond the end of the lumen of the coupling causes expansion of the plug such that it cannot be removed.

According to another example embodiment, as depicted in FIGS. 10-11, a male to female coupling 300 can be provided with additional engagement features for additional engagement between the coupling 300 and the enteral fluid delivery connector VC.

In example embodiments, the coupler 300 is generally similar to the coupler 10 and comprises a first end 312 having a male hub 320 and outer cylindrical collar 323 defining an annular space or fluid containment area 330 and terminating at an endwall, so as to only make the annular space 330 accessible from the first end 312. The second end 314 comprises a female connector comprising a generally cylindrical body 340, and a receiver 342 is defined within the cylindrical body 340 and in fluid communication with the lumen 316 of the hub 320. One or more engagement features such as a rib, lug or thread 344 can be provided on an outer surface of the cylindrical body 340 for engagement with the threaded outer collar W of the enteral fluid delivery connector VC.

In example embodiments, one or more resilient fingers or clips 360 provide for secondary engagement with the enteral fluid delivery connector VC. For example, a pair of oppositely opposed clips 360 (comprising end protrusions extending inwardly) are provided at the second end 314 of the coupling 300, for example, such that when the receiver 342 is fully engaged with the hub H of the enteral fluid delivery connector VC, the clips 360 generally releasably engage an outer portion of the enteral fluid delivery connector VC, for example, wherein the protrusions or projections 362 engage with a grooved surface GS of the groove G formed along an outer body portion of the enteral fluid delivery connector VC. Thus, in example embodiments, the coupling 300 preferably provides for a dual engagement or locking feature wherein a first engagement is provided between lugs 344 of the cylindrical collar 340 of the threaded collar W of the enteral fluid delivery connector VC, and a second engagement is provided between the clips 360 and an outer portion of the feeding tube connector C (e.g., the grooved surface GS of the groove G according to one example embodiment). Optionally, other clips or other complementary coupling features can be provided as desired, for example, such that an additional point of engagement can be provided between the coupling 300 and the enteral fluid delivery connector VC.

In some example embodiments, the clips 360 can be configured for removable engagement wherein a user can press or actuate portions thereof to release the clips from engagement with the enteral fluid delivery connector VC. Optionally, the clips can be configured to permanently engage with the enteral fluid delivery connector VC.

According to another example embodiment, the clips or other engagement features can be provided such that axial and/or rotational displacement between the coupling 300 and the feeding tube connector C causes the clips or other coupling features to disengage with the feeding tube connector. In some example embodiments, a sleeve or skirt 350 projects from the body of the coupling (e.g., from the collar 323 or endwall) towards the second end 314 and a channel formed in the skirt 350 can provide complementary engagement with one or more protrusions formed along an outer periphery portion of the enteral fluid delivery connector VC.

In example embodiments, the skirt comprises a generally cylindrical body 352 defining an outer surface 354, an inner surface 356, and the clips 360 (and projections 362) are formed and integral with the cylindrical body 352. Optionally, the clips, projections and/or other engagement features can be a separate piece assembled with the coupler, or for example, can be co-molded therewith or over molded thereon. In example embodiments, at least a portion of the skirt 350 surrounds the cylindrical body 340. According to the depicted example embodiment, the skirt 350 surrounds the entirety of the cylindrical body 340.

According to example embodiments, while the skirt 350 provides additional engagement features for engagement with the enteral fluid delivery connector VC, the skirt 350 preferably provides a shield and protects the annular space of the enteral fluid delivery connector VC (e.g., from any unused fluids or other contaminants), and thus, provides an additional barrier so as to maintain the enteral fluid delivery connector VC (and annular space thereof) entirely clean and aseptic, and free from any bacteria or harmful contaminants.

According to some example embodiments, the coupling 300 can comprise the skirt 350 for shielding any fluids from entering the annular space of the enteral fluid delivery connector VC; however, the additional engagement features need not be present. In some example embodiments, the skirt 350 is substantially uniform and comprises one or more raised protrusions projecting from an internal surface thereof, for example, for generally sealing with an outer periphery for outer surface of the enteral fluid delivery connector VC. According to some example embodiments, the skirt 350 is generally sized to surround the annular space of the enteral fluid delivery connector VC, however, the skirt 350 need not engage the enteral fluid delivery connector VC (or portions thereof). For example, according to one example embodiment, the skirt 350 comprises an inner diameter that is at least partially larger than an outer side-to-side dimension of the enteral fluid delivery connector VC.

As described above and depicted in FIG. 10, an outer surface or periphery of the outer cylindrical collar 323 can comprise one or more surface features or other recesses, indentions, projections or other features such that an enhanced gripping surface can be provided to assist a user in attachment/detachment of the coupling 300 to/from the connector SC or syringe S. According to the depicted example embodiment, a cylindrical array of longitudinal ribs 324 extend along the outer periphery of the outer cylindrical collar 323, for example, to provide a roughened or texturized outer gripping surface.

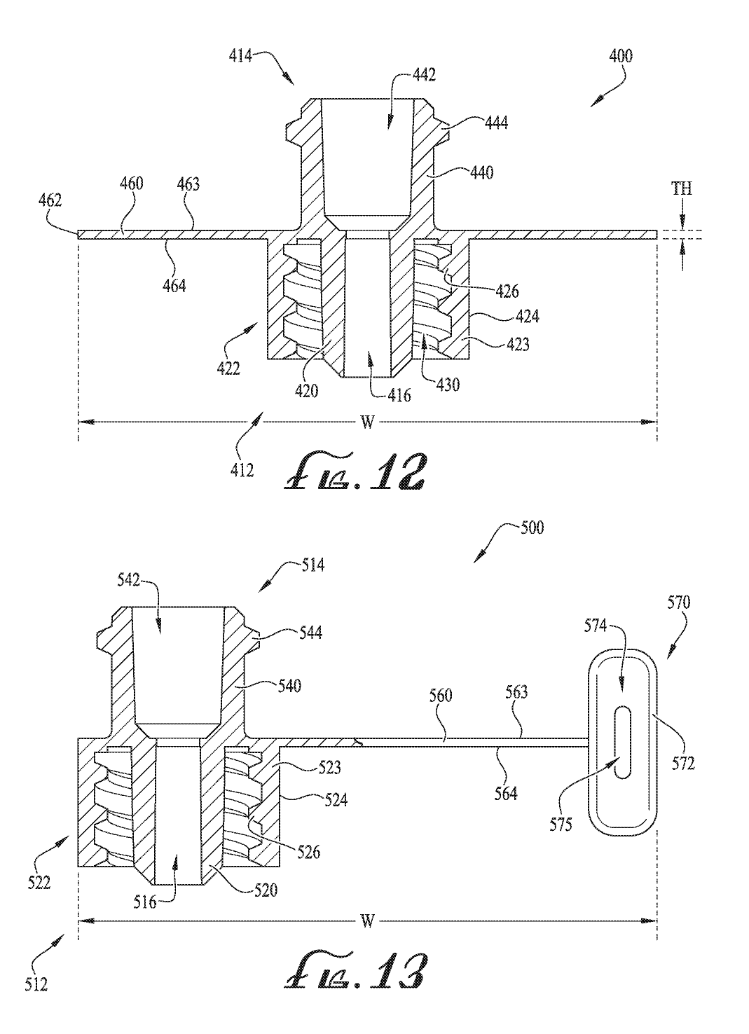

FIGS. 12-13 show couplers 400, 500 according to additional example embodiments of the present invention. In example embodiments, the couplers 400, 500 are generally similar to the couplers as described above, for example, comprising a body extending between a first end 412, 512 and a second end 414, 514. The first end comprises a male hub 420, 520 defining a lumen 416, 516 extending therethrough, and an outer cylindrical collar 423, 523 surrounds the male hub 420, 520 and can comprise threads 426, 526 on an internal surface thereof. As described above, an annular space 430, 530 is provided between the male hub 420, 520 and the outer cylindrical collar 423, 523, which can be utilized to collect any unused fluids that may be present during post fluid delivery disconnection of the first end 412, 512 and the syringe connector SC. The second end 414, 514 comprises a female connector defining a cylindrical body 440, 540, a receiver 442, 542 defined therein and in communication with the lumen 416, 516 of the hub 420, 520. Optionally, one or more lugs or ribs 444, 544 can be formed on an outer surface of the cylindrical body 440, 540.

As depicted in FIG. 12, a disc-like flange 460 extends outwardly from the body or outer cylindrical collar 423 to define a side-to-side dimension W. And as depicted in FIG. 13, a tab 570 extends from the body or collar 523 to define a side-to-side dimension W. According to some example embodiments, to ensure the coupling 10 is not a choking hazard and to comply with 16 CFR § 1501.4, the minimum side-to-side dimension W is about 1.25 inches. According to some example embodiments, the width W is 1.25 inches. According to some example embodiments, the flange 460 comprises a thickness TH of about 0.25 inches. According to other example embodiments, the thickness TH is between 0.01-0.5 inches. According to one example embodiment, the thickness TH is between 0.025-0.15 inches. According to another example embodiment, the thickness TH is between 0.05-0.2 inches.

As depicted in FIG. 12, the flange 460 can be shaped and sized as desired. According to one example embodiment, the flange 460 is substantially uniform to define a substantially cylindrical shape. In some example embodiments, one or more projections or engagement features can be provided on an upper surface 463, a lower surface 464, or an end portion 462 of the flange 460. According to one example embodiment, one or more openings can be provided in the flange 460. According to one example embodiment, an opening is provided for receiving the handle 274 of the cap 270. Thus, the coupler 400 can become tethered to the cap 270 before and/or after use. In other example embodiments, the flange 460 can be shaped as desired, for example, oval, triangular, undulating outer periphery of recesses and ridges, or other linear or non-linear shapes and/or surfaces as desired.

And as depicted in FIG. 13, the tab 570 comprises a generally rectangular body 572 comprising a central opening 574 defining a receiver 575. According to some example embodiments, the tab 570 can similarly be attached to a portion of the cap 270 (e.g., the handle according to some example embodiments) such that the coupler 500 becomes tethered to the enteral fluid delivery connector VC. Preferably, a flange or tether 560 connects the tab 570 with the coupling 500, and which can comprise a desired thickness. In example embodiments, one or more outer surfaces 563, 564 of the tether 560 can comprise one or more projections, openings or other engagement features as desired. According to some example embodiments, the tab 570 is sized and configured for passing through the lumen 516, for example, such that the tab 570 becomes caught and unable to remove itself, thereby providing a reuse prevention feature so that the coupling is not accidentally used more than once. According to some example embodiments, even when the tab 570 is pushed through the lumen 516, the tether 560 still extends sufficiently outwardly such that a side-to-side dimension of 1.25 inches is still provided. According to one example embodiment, the body 572 of the tab 570 is preferably resiliently flexible, for example, such that it can be deformed to allow passage through the lumen 516.

Optionally, according to some example embodiments, one or more clips can provide for removable or permanent engagement with the syringe connector SC of the syringe S, for example, wherein the outer threaded outer cylindrical collars of the couplings 10, 100, 200, 300, 400, 500 described herein can be modified to provide for dual-action attachment and removal with the syringe connector SC of the syringe S. Optionally, the outer cylindrical collar of the enteral fluid delivery connector VC can similarly comprise a modified or clipped collar. U.S. patent application Ser. No. 15/454,761 (Attorney Docket No. 2N11.1-410), U.S. patent application Ser. No. 15/078,674 (Attorney Docket No. 2N11.1-322), U.S. patent application Ser. No. 15/185,583 (Attorney Docket No. 2N11.1-351), U.S. patent application Ser. No. 14/844,922 (Attorney Docket No. 2N11.1-313), U.S. Design patent application Ser. No. 29/521,665 (Attorney Docket No. 2N11.1-300), and U.S. Design patent application Ser. No. 29/533,173 (Attorney Docket No. 2N11.1-301) are incorporated herein by reference and disclose various clipped, snap-on and dual-action attachment and removal mechanisms for replacement with the outer coaxial connection collars of the male to female couplings 10, 100, 200, 300, 400, 500 and/or the enteral fluid delivery connectors C, VC.

According to another example embodiment, the present invention relates to a method of enteral feeding comprising providing a fluid delivery device. In example embodiments, the method comprises providing an enteral feeding tube assembly, the assembly comprising an elongate tube comprising at least one connector; providing a male to female coupling, the male to female coupling comprising a body extending along a longitudinal axis from a first end to a second end; connecting the second end of the male to female coupling with the at least one connector of the enteral feeding tube assembly; and connecting the first end of the male to female coupling with the fluid delivery device.

According to another example embodiment, the present invention relates to a method of connecting and disconnecting enteral feeding connectors to prevent contamination of components. In example embodiments, the method comprises providing a fluid delivery device, the fluid delivery device comprising a connector; providing an enteral feeding tube assembly, the assembly comprising an elongate tube comprising at least one connector; providing a male to female coupling, the male to female coupling comprising a body extending along a longitudinal axis from a first end to a second end, a hub and an outer cylindrical collar defined at the first end and a receiver comprising a cylindrical body at the second end, wherein an annular space is defined between the hub and the outer cylindrical collar, the annular space terminating at an endwall and the hub being configured for compatible engagement with the fluid delivery syringe, and wherein a lumen extends through the cylindrical body and hub so as to provide fluid communication through the body, the receiver of the second end configured for compatible engagement with the at least one connector of the enteral feeding tube assembly; connecting the receiver of the male to female coupling with the at least one connector of the enteral feeding tube assembly; and connecting the hub of the male to female coupling with the connector of the fluid delivery device; discharging fluid from the fluid delivery device, through the lumen of the male to female coupling, and through the conduit of the enteral feeding tube assembly; disconnecting or separating the connector of the fluid delivery device from the hub of the male to female connector, wherein any fluids overflowing from the lumen of the hub of the male to female coupling are retained within the annular space; and disconnecting the receiver of the second end of the male to female coupling from the at least one connector of the enteral feeding tube assembly.

While the invention has been described with reference to example embodiments, it will be understood by those skilled in the art that a variety of modifications, additions and deletions are within the scope of the invention, as defined by the following claims.

What is claimed is:

1. A male to female enteral coupling comprising:
a body extending from a first end to a second end, the first end comprising a male hub surrounded by an outer cylindrical collar, an annular space defined between the male hub and the outer cylindrical collar, and a lumen defined within the male hub, and the second end comprising a female connector comprising a generally cylindrical body, and a receiver defined within the body and in communication with the lumen, the receiver configured for receiving a male connector,
wherein the annular space is configured for collecting fluids discharged or overflowing during a post-delivery disconnection or separation of a fluid delivery syringe and an enteral fluid delivery device connector.

2. The male to female enteral coupling of claim 1, wherein the male to female coupling is configured for interconnection between the fluid delivery syringe and an enteral fluid delivery device connector.

3. The male to female enteral coupling of claim 1, wherein the first end is configured for engagement with a syringe connector of the fluid delivery syringe and the second end is configured for engagement with a male hub of the enteral fluid delivery device connector.

4. The male to female enteral coupling of claim 1, wherein the first end comprises a male ISO 80369-3 compatible coupling.

5. The male to female enteral coupling of claim 1, wherein the second end comprises a female ISO 80369-3 compatible coupling.

6. The male to female enteral coupling of claim 1, further comprising a flange extending from at least a portion of the body.

7. The male to female enteral coupling of claim 6, wherein the flange comprises an outwardly-protruding member, and wherein a width defined between the body an outermost portion of the outwardly-protruding member is a minimum of about 1.25 inches.

8. The male to female enteral coupling of claim 1, further comprising a tether for maintaining engagement of the body with the enteral fluid delivery device connector.

9. The male to female enteral coupling of claim 1, further comprising a lumen extension tip centrally positioned within the receiver of the cylindrical body, the lumen extension tip comprising a conduit defined therein and in communication with the lumen of the male hub of the first end.

10. The male to female enteral coupling of claim 1, further comprising a skirt projecting from the body and towards the second end, at least a portion of the skirt surrounding the cylindrical body of the second end.

11. The male to female enteral coupling of claim 10, further comprising one or more resilient clips formed with at least a portion of the skirt.

12. A disposable enteral fluid delivery connector for interconnection between a fluid delivery syringe and a connector of an enteral feeding tube, the disposable enteral fluid delivery connector comprising:
a body extending along a longitudinal axis from a first end to a second end;
a hub and an outer cylindrical collar defined at the first end, and an annular space defined between the hub and the outer cylindrical collar, the annular space terminating at an endwall, and the hub being configured for compatible engagement with the fluid delivery syringe;
a receiver comprising a cylindrical body at the second end, and a lumen extending through the cylindrical body and hub so as to provide fluid communication through the body, the receiver configured for compatible engagement with the connector of the enteral feeding tube,
wherein, with the body interconnected between the fluid delivery syringe and the connector of the enteral feeding tube, the fluid delivery syringe is disconnected from the hub of the first end such that any fluids discharged from the lumen of the hub or from the fluid delivery syringe is collected in the annular space defined at the first end of the body so as to prevent exposure of fluids to the connector of the enteral feeding tube.

13. The male to female coupling of claim 12, wherein the first end comprises a male ISO 80369 compatible coupling.

14. The male to female coupling of claim 12, wherein the second end comprises a female ISO 80369 compatible coupling.

15. The male to female coupling of claim 12, further comprising a flange extending from at least a portion of the body.

16. The male to female coupling of claim 16, wherein the flange comprises an outwardly-protruding member, and wherein a width defined between the body and an outermost portion of the outwardly-protruding member is a minimum of about 1.25 inches.

17. The male to female coupling of claim 12, further comprising a tether for maintaining engagement of the body with the enteral fluid delivery device connector.

18. The male to female coupling of claim 12, further comprising a lumen extension tip centrally positioned within the receiver of the cylindrical body, the lumen extension tip comprising a conduit defined therein and in communication with the lumen of the male hub of the first end.

19. The male to female coupling of claim 12, further comprising a skirt projecting from the body and towards the second end, at least a portion of the skirt surrounding the cylindrical body of the second end.

20. The male to female coupling of claim 19, further comprising one or more resilient clips formed with at least a portion of the skirt, the one or more resilient clips being configured for removable or permanent engagement with the connector of the enteral feeding tube.

21. A method of connecting and disconnecting enteral feeding connectors to prevent contamination of components, the method comprising:
providing a fluid delivery device, the fluid delivery device comprising a connector;
providing an enteral feeding tube assembly, the assembly comprising an elongate tube comprising at least one connector;
providing a male to female coupling, the male to female coupling comprising a body extending along a longitudinal axis from a first end to a second end, a hub and an outer cylindrical collar defined at the first end and a receiver comprising a cylindrical body at the second end, wherein an annular space is defined between the hub and the outer cylindrical collar, the annular space terminating at an endwall and the hub being configured for compatible engagement with the fluid delivery syringe, and wherein a lumen extends through the cylindrical body and hub so as to provide fluid communication through the body, the receiver of the second end configured for compatible engagement with the at least one connector of the enteral feeding tube assembly;
connecting the receiver of the male to female coupling with the at least one connector of the enteral feeding tube assembly; and
connecting the hub of the male to female coupling with the connector of the fluid delivery device;
discharging fluid from the fluid delivery device, through the lumen of the male to female coupling, and through the conduit of the enteral feeding tube assembly;
disconnecting or separating the connector of the fluid delivery device from the hub of the male to female connector, wherein any fluids overflowing from the lumen of the hub of the male to female coupling are retained within the annular space; and
disconnecting the receiver of the second end of the male to female coupling from the at least one connector of the enteral feeding tube assembly.
